(19) United States
(12) Patent Application Publication
(10) Pub. No.: US 2011/0245627 A1
(43) Pub. Date: Oct. 6, 2011

(54) ELECTRONIC HEALTH RECORD STORAGE DEVICE, SYSTEM, AND METHOD

(75) Inventors: Lydia Lap Wai Leung, Hong Kong (CN); Luis Ng, Hong Kong (CN); Kwan Wai To, Hong Kong (CN); Chi Tin Luk, Hong Kong (CN); Chang Hwa Chung, Hong Kong (CN)

(73) Assignee: Hong Kong Applied Science and Technology Research Institute Co., Ltd., Hong Kong (CN)

(21) Appl. No.: 12/749,906

(22) Filed: Mar. 30, 2010

(51) Int. Cl. A61B 5/00; G06Q 50/00; G06Q 10/00; G06F 13/10

(52) U.S. Cl. 600/301; 705/2; 710/5; 710/301

(57) ABSTRACT

An electronic health record storage device is provided. The device is configured for operation in conjunction with a host device to generate and store health record data about a user. According to one embodiment, the system comprises memory including executable instructions stored in the memory, the executable instructions including an autorun file and operating instructions; one or more sensors configured for engagement by the user; an interface configured for interfacing with a host device; and a controller in operable communication with the memory, the one or more sensors, and the interface, and wherein the controller is configured to execute the executable instructions stored in the memory, the controller further configured to receive power from the host device, retrieve data from the host device, receive health data from the one or more sensors, generate a health record comprising timing data and health data, and store the health record in the memory.

### FIELD OF THE INVENTION

The present invention relates to an electronic health record storage device, and more particularly, to a device, system and method for generating and storing health records.

### BACKGROUND OF THE INVENTION

With the advent of the digital age, the ability to store and access data electronically has changed and simplified the way companies do business. In the health care industry, medical records and medical information are also moving in the direction of the technology age. Many hospitals and companies now keep only electronic files on their customers and patients. Increasingly, electronic health records (EHR), or electronic patient records, are being used to improve the quality of the health services provided.

An EHR is generally a collection of electronic health information about an individual patient or a group of people. The health information is recorded in a digital format that is capable of being shared between or among people that have the need to access the particular information. The EHR may include any kind of data in either a comprehensive or summary form, including demographics, medical history, medication and allergies, immunization status, laboratory test results, radiology images, and billing information or any other relevant information.

Typically, an EHR is generated and maintained within an institution, such as a hospital, medical clinic, or physician's office. However, currently there exist some devices designed to generate health records outside of these locations. Some of the existing devices require complex systems, continuous connection to a source of power, or a user's ability to operate a complex computer system. These existing devices can prove not to be user-friendly, especially to people without computer knowledge.

Accordingly, there is a need for an electronic health record storage device, system and method that overcomes these and other shortcomings of known devices.

### SUMMARY OF THE INVENTION

According to one embodiment of the present invention, an electronic health record storage device configured for operation in conjunction with a host device to generate and store health record data on a user is disclosed. The electronic health record storage device includes memory including executable instructions stored in the memory, the executable instructions including an autorun file and operating instructions; one or more sensors configured for engagement by the user; an interface configured for interfacing with a host device; and a controller in operable communication with the memory, the one or more sensors, and the interface, and wherein the controller is configured to execute the executable instructions stored in the memory, the controller further configured to receive power from the host device, retrieve data from the host device, receive health data from the one or more sensors, generate a health record comprising timing data and health data, and store the health record in the memory.

According to another embodiment of the present invention, a method for generating and storing an electronic health record on a storage device. The method includes connecting the storage device with a host device; receiving power from the host device; receiving timing information from the host device; executing operating instructions on the storage device; receiving measurements of the user's health; generating health record data, the health record data including a representation of the measurements and the timing information; and storing the health record data in a storage device memory.

Still other embodiments of the present invention will become readily apparent to those skilled in the art from the following detailed description, wherein embodiments of the invention are described by way of illustration. As will be realized, the invention is capable of other and different embodiments and its several details are capable of modifications in various respects, all without departing from the spirit and the scope of the present invention.

### BRIEF DESCRIPTION OF THE DRAWINGS

FIG. 1 is a block diagram of an electronic health record storage device and a host device, in accordance with an embodiment of the present invention.

FIG. 2 is an electronic health record storage device and an example host device, in accordance with an embodiment of the present invention.

FIG. 3 is a detailed, top view of the electronic health record storage device shown in FIG. 2, in accordance with an embodiment of the present invention.

FIG. 4 is a side cross section view of the electronic health record storage device shown in FIG. 3, in accordance with an embodiment of the present invention.

FIG. 5A is a flowchart diagram of an example host device process, in accordance with an embodiment of the present invention.

FIG. 5B is a flowchart diagram of an example storage device process, in accordance with an embodiment of the present invention.

FIG. 6 is an electronic health record storage device having a separable extension, in accordance with an embodiment of the present invention.

### DETAILED DESCRIPTION

In the following description, reference is made to the accompanying drawings where, by way of illustration, specific embodiments of the invention are shown. It is to be understood that other embodiments may be used as structural and other changes may be made without departing from the scope of the present invention. Also, the various embodiments and aspects from each of the various embodiments may be used in any suitable combinations. Accordingly, the drawings and detailed description are to be regarded as illustrative in nature and not as restrictive. Like elements in each of the figures are referred to by like reference numbering.

Generally, embodiments of the present invention are directed to an electronic health record storage device and a system and method of generating and storing health record data. According to one embodiment, the electronic health record storage device includes a controller, memory, and one or more sensors for measuring the vital signs, or other information, of the user of the electronic health record storage device. According to one embodiment, the electronic health record storage device has the shape and interface of a conventional memory card or memory device such that it can be used with conventional electronic devices, such as computers, mobile telephones, personal data assistants, and similar electronic devices. After electronic health records are generated and stored on the electronic health record storage device, a doctor or other user may view or copy the health records to use them in connection with an assessment of the user's health, with the authorization of the owner of the storage device.

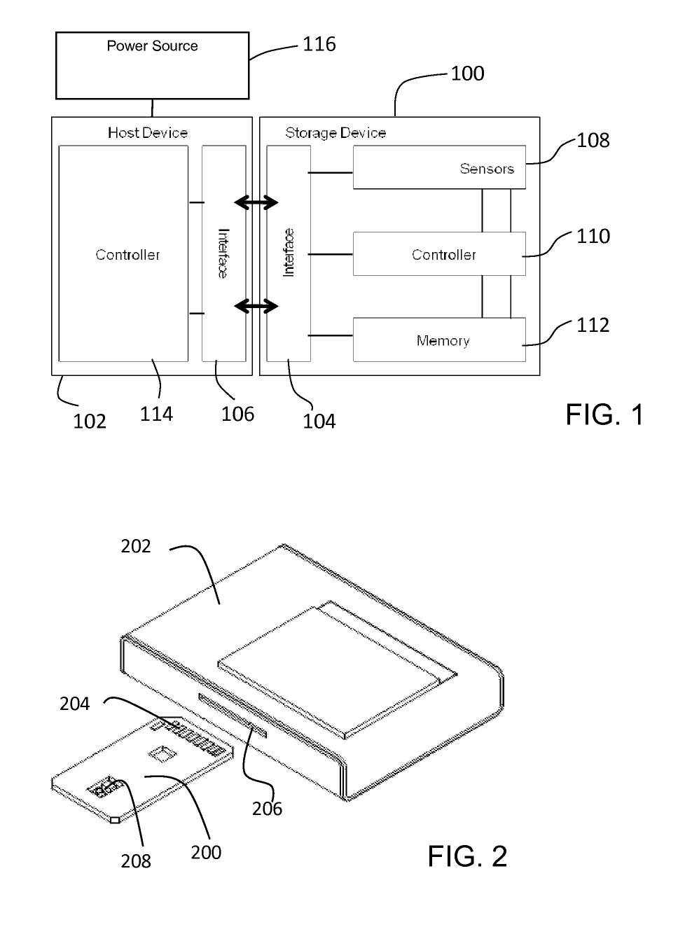

Referring now to the figures, FIG. 1 is a block diagram of the electronic health record storage device, also referred to as “the storage device”, and the host device, in accordance with an embodiment of the present invention. The storage device 100 is configured for use with the host device 102. The storage device 100 includes a storage device interface 104 configured for connection to a host device interface 106. The host device interface 106 is attached or coupled to the host device 102. The storage device 100 further includes one or more sensors 108, a storage device controller 110, and memory 112. The host device 102 includes a host device controller 114. In one embodiment of the present invention, the host device 102 is an electronic device, such as a computer, laptop, mobile telephone, personal data assistant, electronic tablet, portable gaming system, or other similar electronic device having a power source 116. For example, the power source may be AC or DC current, such as by plugging into an electric circuit or being powered by one or more batteries. According to one embodiment of the present invention, the storage device 102 does not include a power source and during operation receives power from the host device through the storage device interface 104.

FIG. 2 is an example electronic health record storage device 200 and an example host device 202, in accordance with an embodiment of the present invention. The storage device 200 illustrated in FIG. 2 is a standard memory card, such as an SD card or a micro SD card. However, other types of memory devices may be used. The storage device 200 has a memory card interface 204 integrated with the storage device 200. The host device 202 is an example electronic device with a memory card slot 206 configured to receive the storage device. While not shown in the illustrated embodiment, the host device 202 includes a host device interface in the memory card slot 206 that will connect with the memory card interface 204 during operation when the storage device 200 is inserted into the memory card slot 206. The storage device 200 includes one or more sensors 208 for measuring certain user information, such as the user's vital signs. For example, the one or more sensors 208 may be referred to as vital sign measurement (VSM) sensors and are configured to measure such user information as the user's pulse, temperature, blood-oxygen saturation, and electrical activity of the heart. Some example sensors include, for example, an electrocardiography sensor and a pulse oximeter.

According to one embodiment, the one or more sensors 208 are built into the storage device 200. However, according to another embodiment, the one or more sensors 208 may also be assembled on the surface of the storage device 200 or embedded into the memory card. According to one embodiment, the overall thickness of the storage device 200 remains unchanged such that the inclusion of the one or more sensors 208 doesn't interfere with ability of the storage device 200 to be used with conventional, commercially available host devices 202 that are configured to receive conventional storage devices that are similar to the storage device 200 made according to embodiments of the present invention.

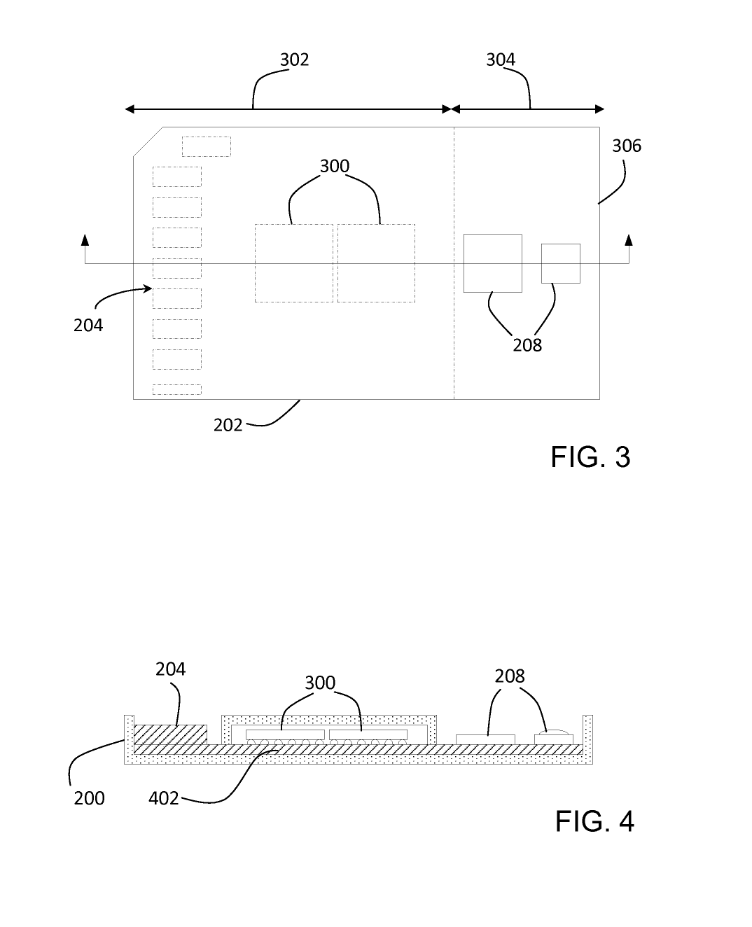

Referring to FIGS. 3 and 4, FIG. 3 is a detailed, top view of the electronic health record storage device shown in FIG. 2, and FIG. 4 is a side cross section view of the electronic health record storage device shown in FIG. 3 taken at line A, in accordance with an embodiment of the present invention. The storage device 200 includes the memory card interface 204 and the one or more sensors 208. Also, a memory card circuitry 300 is shown in the illustrated embodiment. The memory card circuitry 300 may be embedded within the storage device 200 and include the necessary electronic components of the storage device 204, such as the controller 110 and the memory 112 and any other necessary components, such as filters or amplifiers. As shown in FIG. 3, a first arrow 302 indicates a length of the standard memory card, and a second arrow 304 indicates a length of an extension 306 of the storage device 200. Therefore, since a standard memory card is generally inserted completely into the host device 202 in use, the extension 306 of the storage device will protrude from the host device 202 such that the user may access and engage the one or more sensors 208 that are positioned on the extension 306 of the storage device 200. According to one embodiment, the extension 306 is contiguously formed with the storage device 200 and is a part of the storage device 200. As shown in FIG. 4, a printed circuit board 402 is used to connect the memory card interface 204, the memory card circuitry 300, and the one or more sensors 208. One or more circuit boards may be included according to the desired design of the storage device being used.

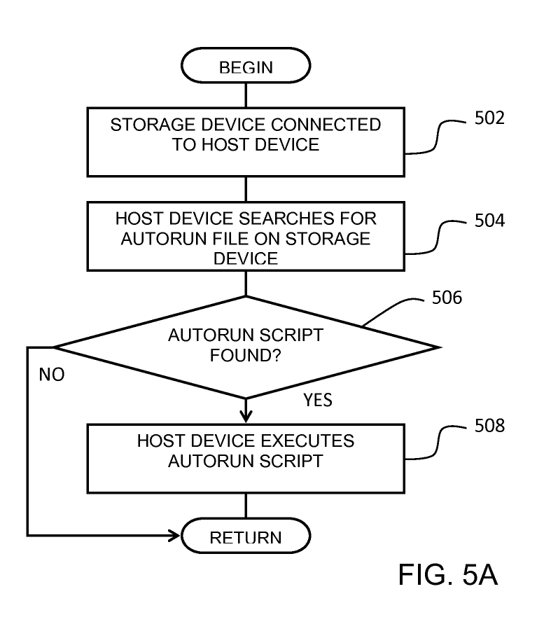

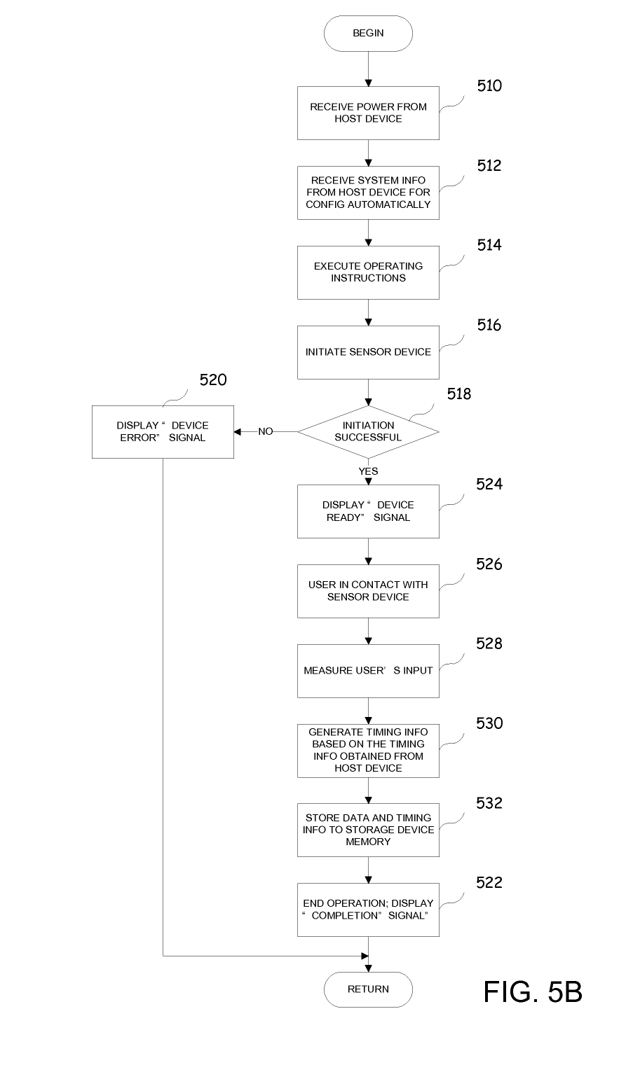

FIG. 5A is a flowchart diagram of an example host device process, in accordance with an embodiment of the present invention. In step 502, the storage device is connected to the host device. In one embodiment, the storage device will be a memory card inserted into the memory card slot of the host device. In step 504, the host device searches for an autorun file stored on the storage device. In step 506, the host device will determine if the autorun file is found. If not, then the process ends. If yes, then the host device executes the autorun file, step 508. According to one embodiment of the present invention, the autorun file contains instructions that allow the storage device to operate and access host device resources. For example, the autorun file includes instructions for transferring the timing information from the host to the storage device. Other resources include any other data that may be useful for the future data analysis of the data stored in the storage device. Once the storage device receives power, the storage device executes its own operating program or functions. According to one embodiment, the storage device executes the operation of the storage device using the storage device components, including the controller and the memory of the storage device. Therefore, throughout the operation of the storage device, the host device provides the power supply and the storage device is effectively transparent to the host device.

FIG. 5B is a flowchart diagram of an example storage device process, in accordance with an embodiment of the present invention. After successful completion of the process illustrated and described with reference to FIG. 5A, the storage device executes the process shown and illustrated with reference to FIG. 5B. If the autorun file is not executed by the host device, step 508 in FIG. 5A, then the process of FIG. 5B may still be properly executed and the measurement results may be stored without the timing information.

In step 510, the storage device receives power from the host device. In step 512, the storage device obtains information from the host device for configuration of the storage device automatically. This may be done by the host device running the autorun file described with reference to step 504 in FIG. 5A. In step 514, the storage device executes operating instructions. The operating instructions are stored in the memory of the storage device. Therefore, the storage device is configured to process the executable instructions. In step 516, the storage device initiates the one or more sensor devices. In step 518, the storage device determines if the initiation was successful. If not, then the storage device will display a “device error” signal to the user and return to a waiting stage. In the waiting stage, the user may wait until the initiation completes and is successful, or the process may end and require the user to reinsert the storage device. If the process is ended, then the storage device will proceed to step 522, the storage device will display a “completion” signal, and the storage device may be removed from the host device. According to one embodiment, the signals may be, for example, a light emitting diode or other light signal, and different colors of the light emitting diode will be different signals, or two or more light emitting diodes may be used to convey different meanings. If the initiation is successful, the process continues to step 524 and the storage device displays a “device ready” signal to the user. At this point, the user must engage the one or more sensors on the storage device. Then, at step 526, after the user has engaged the one or more sensors, the storage device receives input from the user. Depending on the type of sensor or sensors being used, the input received by the storage device may be different. For example, if the sensor is configured to detect a user's pulse, the input may be vibrations cause by blood flow in the user's thumb or another part of the user's body. In step 528, the storage devices measures the user's input as necessary and configures the input into data that can be stored on the storage device memory. In step 530, the storage device calculates the timing information based on the system information collected from the host device. The timing data may be the time and the date at which the process was executed. The timing data may also be any other suitable information that indicates when this specific occurrence of measurements was taken. Also, according to another embodiment, the timing data may also be obtained from an atomic clock or radio clock. The timing data does not need to be retrieved in this order in the process but may be retrieved at any other time in the process. In step 532, data is stored to the storage device memory. The data stored to the storage device memory is a data record that may be accessed at a later time by a suitable host device configured to open and view the data record. The data may include both the measurements of the user's input taken in step 528 as well as the timing data. While illustrated as a single step, different parts of the data may also be saved to the storage device memory in multiple steps at multiple different steps in the process, according to the desired implementation.

The processes illustrated and described with reference to FIGS. 5A and 5B may be repeated multiple times with the same storage device. Accordingly, by repeating the processes with the same storage device, multiple data records will be stored on the same storage device. These multiple data records will comprise measurements of a particular user at different periods of time. A doctor, for example, or other person may then insert the storage device into a suitable host device to open and view the data records. Accordingly, embodiments of the present invention may be used to generate an electronic health record stored on the memory of a storage device. In the embodiment where a standard memory card is used, the standard memory card may be connected to any suitable computer or host device having a compatible interface, and the data records may be viewed, copied, or otherwise manipulated using any suitable file management application.

According to one embodiment of the present invention, the storage device generates an electronic health record of the user without using an application programming interface (API). Instead, the storage device operates according to the described process to obtain and store user data on the storage device memory.

Accordingly, one embodiment of the present invention provides for an EHR storage device having sensors integrated into the storage device, which may be configured to have the size, shape and interface of a standard memory card or external memory device. Therefore, during use, the user only needs to carry the storage device with them. When it's time to make a health measurement, the user can insert the storage device into any host device having a compatible interface, and the storage device will obtain the information that it needs to take a measurement and store the measurement data onto the storage device from the host device. Embodiments of the present invention can increase the convenience of the user since the storage device is generally small and compact and it may not be necessary to bring any other equipment. The storage device can be used with any suitable host devices, which can be a device that is commonly carried by many users, such as a mobile telephone or other electronic device. According to one embodiment, the storage device obtains the necessary information from the host device automatically without using an API and with an interface that is user friendly and useable by those without knowledge of computer data applications. The storage device can be given to the user's physician, who can transfer the measurement to his computer by connecting the storage device to his computer having a compatible interface.

While the embodiments of the present invention may be implemented as executable instructions stored on the memory, the functions and operations required for implementing embodiments of the invention may also be embodied in whole or in part using hardware components, such as application specific integrated circuits or other suitable hardware, or some combination of hardware components and computer executable instructions. The controller of the storage device may be any suitable microcontroller or processing unit. The controller of the host device may also be any suitable microcontroller or processing unit.

In the illustrated embodiments shown in FIGS. 2 to 4, the one or more sensors 208 are located on an extension of the storage device 200, the extension 306 being generally contiguous with the storage device 200. However, embodiments of the present invention may include the extension 306 separated from the storage device 200 if necessary for the particular application.

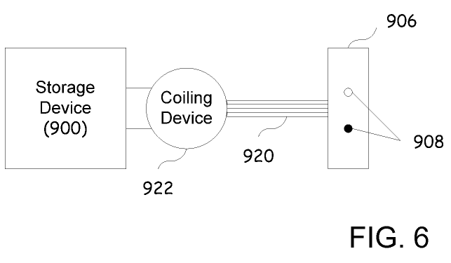

FIG. 6 is an electronic health record storage device having a separable extension, in accordance with an embodiment of the present invention. According to the embodiment of the present invention illustrated in FIG. 9, the extension 906 having the one or more sensors 908 is separated from, or separable from, the storage device 900, and the extension 906 is connected to the storage device 900 by a cable 920. In the illustrated embodiment, the cable 920 is included with a coiling device 922 that is configured to hold and retract the cable 920 for the convenience of the user. Therefore, the extension and the one or more sensors 908 are separably connected to the storage device 900 by the cable 920.

The coiling device may be any suitable structure, such as a structure including a housing, a rotatable spool, and a spring. The cable is wound onto the rotatable spool, and the spring generates force to rotate the spool and wind up the cable. When the cable is pulled, the cable is released from the housing, and the rotation force will also be released. Any other conventional retraction or elastic devices may be used.

Using a separable or separated sensor, embodiments of the present invention can be more easily used by a user of the present invention. Also, the separated configuration may reduce the amount of interference generated by the host device, such as light interference or electromagnetic interference generated by the host device, such as when the host device is a device having wireless communications capabilities, such as a mobile telephone.

While the invention has been particularly shown and described with reference to the illustrated embodiments, those skilled in the art will understand that changes in form and detail may be made without departing from the spirit and scope of the invention. For example, while the steps illustrated and described with reference to FIGS. 5A and 5B are in a specified order, it is to be understood that these steps may also be performed in a different order. Also, these steps are only one example process and, in accordance with other embodiments, additional steps may be included in the process and/or one or more included steps may be omitted, as needed by a particular implementation of the present invention. Also, while an example storage device and host device are illustrated and described with reference to FIGS. 2 to 4, other suitable devices may be used.

Accordingly, the above description is intended to provide example embodiments of the present invention, and the scope of the present invention is not to be limited by the specific examples provided.

What is claimed is:

1. An electronic health record storage device configured for operation in conjunction with a host device to generate and store health record data of a user, the electronic health record storage device comprising:
memory including executable instructions stored in the memory, the executable instructions including an autorun file and operating instructions;
one or more sensors configured for engagement by the user;
an interface configured for interfacing with a host device; and
a controller in operable communication with the memory, the one or more sensors, and the interface, and wherein the controller is configured to execute the executable instructions stored in the memory, the controller further configured to receive power from the host device, retrieve data from the host device, receive health data from the one or more sensors, generate a health record comprising timing data and health data, and store the health record in the memory.

2. The electronic health record storage device of claim 1, wherein the host device retrieves an autorun file from the storage device, and wherein the autorun file includes instructions for the host device to permit the storage device to access host device resources.

3. The electronic health record storage device of claim 1, wherein the storage device is configured to have a size and a shape of a memory card, and the interface is a memory card interface configured for connection to a compatible host device interface.

4. The electronic health record storage device of claim 1, wherein the storage device is configured to have a size and a shape of a memory stick, and the interface is a memory stick interface configured for connection to a compatible host device interface.

5. The electronic health record storage device of claim 3 or 4, wherein the memory card includes an extension, and the one or more sensors are located in the extension.

6. The electronic health record storage device of claim 4, wherein the extension is integrated with the memory card and the one or more sensors are embedded in the extension.

7. The electronic health record storage device of claim 4, wherein the extension is separably and operably connected to the storage device by a cable.

8. The electronic health record storage device of claim 1, wherein the one or more sensors are configured to receive user input from the user in the form of vital signs, and wherein the one or more sensors are configured to the health data comprises information on the user input.

9. The electronic health record storage device of claim 1, wherein the one or more sensors comprise an electrocardiography sensor.

10. The electronic health record storage device of claim 1, wherein the one or more sensors comprise a pulse oximeter.

11. A method for generating and storing an electronic health record on a storage device, the method comprising:
connecting the storage device with a host device;
receiving power from the host device;
receiving timing information from the host device;
executing operating instructions on the storage device;
receiving measurements of the user's health;
generating health record data, the health record data including a representation of the measurements and the timing information; and
storing the health record data in a storage device memory.

12. The method of claim 11, further comprising:
displaying a “device ready” signal to the user;
receiving input from the user; and
displaying a “disconnect” signal from the user.

13. The method of claim 11, further comprising the step of initiating the one or more sensor for use.

14. The method of claim 11, wherein the timing information includes the time and date of the operation of the storage device.

15. The method of claim 11, wherein the measurements are a representation of the user's vital signs.

16. A method for generating and storing an electronic health record on a storage device, the method comprising:
connecting the storage device with a host device;
receiving power from the host device;
receiving timing information from an external source;
executing operating instructions on the storage device;
receiving measurements of the user's health;
generating health record data, the health record data including a representation of the measurements and the timing information; and
storing the health record data in a storage device memory.

17. The method of claim 16, wherein the external source is a wireless communications signal.

18. The method of claim 16, wherein the external source is an atomic clock.

19. The method of claim 16, wherein the external source is a radio clock.
